(19) United States
(12) Patent Application Publication
(10) Pub. No.: US 2026/0041358 A1
(43) Pub. Date: Feb. 12, 2026

(54) DEVICE FOR MONITORING MULTI-CHANNEL BIOSIGNAL

(71) Applicant: HUINNO, CO., LTD., Seoul (KR)

(72) Inventors: Yeong Joon GIL, Busan (KR); Sung Hoon JUNG, Seoul (KR); Eu Bin JEONG, Gunpo-si (KR); Young Soo JANG, Bucheon-si (KR); Seong Bok LEE, Seoul (KR); Won Young LEE, Bucheon-si (KR)

(21) Appl. No.: 19/217,227

(22) Filed: May 23, 2025

Related U.S. Application Data
(63) Continuation of application No. PCT/KR2023/017044, filed on Oct. 30, 2023.

(30) Foreign Application Priority Data
Nov. 24, 2022 (KR) 10-2022-0159694
Apr. 17, 2023 (KR) 10-2023-0050367

(51) Int. Cl. A61B 5/318; A61B 5/0245; A61B 5/257; A61B 5/28

(52) U.S. Cl. CPC A61B 5/318; A61B 5/0245; A61B 5/257; A61B 5/28

(57) ABSTRACT

A device for monitoring a biosignal includes a main body unit; and an electrode unit detachably coupled to the main body unit. The electrode unit includes two or more electrodes for acquiring the biosignal, and a circuit unit for electrically connecting the electrodes and the main body unit.

### CROSS-REFERENCE TO RELATED APPLICATION(S)

This application is a Continuation of International Application No. PCT/KR2023/017044 filed on Oct. 30, 2023, which claims priority from Korean Application No. 10-2022-0159694 filed on Nov. 24, 2022 and Korean Application No. 10-2023-0050367 filed on Apr. 17, 2023. The aforementioned applications are incorporated herein by reference in their entireties.

### TECHNICAL FIELD

The present invention relates to a device for monitoring a multi-channel biosignal.

### RELATED ART

Due to progress in science and technology, various techniques for monitoring biosignals are being developed. Biosignals include not only electrocardiogram (ECG) signals but also various signals that can be collected from a body, such as blood oxygen saturation, heart rate, and body temperature.

Meanwhile, among biosignals, ECG signals can be useful information for determining the presence of heart disease, so that techniques for collecting and monitoring ECG signals are being developed.

Conventional wearable ECG monitoring devices employ predetermined off-the-shelf disposable Ag/AgCl electrodes, or are integrally configured such that electrodes are merged with a main body containing circuitry for ECG monitoring. Therefore, most wearable ECG monitoring devices can only collect ECG signals by connecting a fixed number of electrodes with the same structure, which causes a problem that a heart, which is a three-dimensional structure, cannot be examined in detail because ECG signals cannot be collected by changing the positions of the electrodes or increasing the number of the electrodes.

### SUMMARY

One object of the present invention is to solve all the above-described problems in the prior art.

Another object of the invention is to provide a device for monitoring a biosignal, the device comprising a main body unit and an electrode unit detachably coupled to the main body unit, wherein the electrode unit includes two or more electrodes for acquiring the biosignal and a circuit unit for electrically connecting the electrodes and the main body unit.

Yet another object of the invention is to enable collection of various biosignals and accurate identification of a patient's heart disease by employing an electrode unit including various electrode arrangements or two or more electrodes depending on the patient's condition.

Still another object of the invention is to provide a wearable monitoring device capable of collecting various biosignals in a single main body by allowing various types of electrode units to be coupled to the single main body.

The representative configuration of the invention to achieve the above objects is described below.

According to one aspect of the invention, there is provided a device for monitoring a biosignal, the device comprising a main body unit and an electrode unit detachably coupled to the main body unit, wherein the electrode unit includes two or more electrodes for acquiring the biosignal and a circuit unit for electrically connecting the electrodes and the main body unit.

In addition, there are further provided other devices according to the technical idea of the invention.

According to the invention, it is possible to provide a wearable monitoring device capable of collecting various biosignals and accurately identifying a patient's heart disease by employing an electrode unit including various electrode arrangements or two or more electrodes depending on the patient's condition.

According to the invention, it is possible to provide a wearable monitoring device capable of collecting various biosignals in a single main body by allowing various types of electrode units to be coupled to the single main body.

### BRIEF DESCRIPTION OF THE DRAWINGS

FIG. 1 shows a main body unit 100 and an electrode unit 200 according to one embodiment of the invention.

FIG. 2 shows a main body unit 100 and an electrode unit 200 according to one embodiment of the invention.

FIG. 3 shows a main body unit 100 and an electrode unit 200 according to one embodiment of the invention.

FIG. 4 illustratively shows a situation in which a biosignal is stored separately for each channel according to one embodiment of the invention.

### DESCRIPTION OF THE REFERENCE NUMERALS

10: device
100: main body unit
200: electrode unit
210: electrodes
220: circuit unit
230: coupling unit
240: extension connection unit
250: pad
250: additional pads

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the invention will be described in detail with reference to the accompanying drawings to enable those skilled in the art to easily implement the invention.

### Configuration of a Device

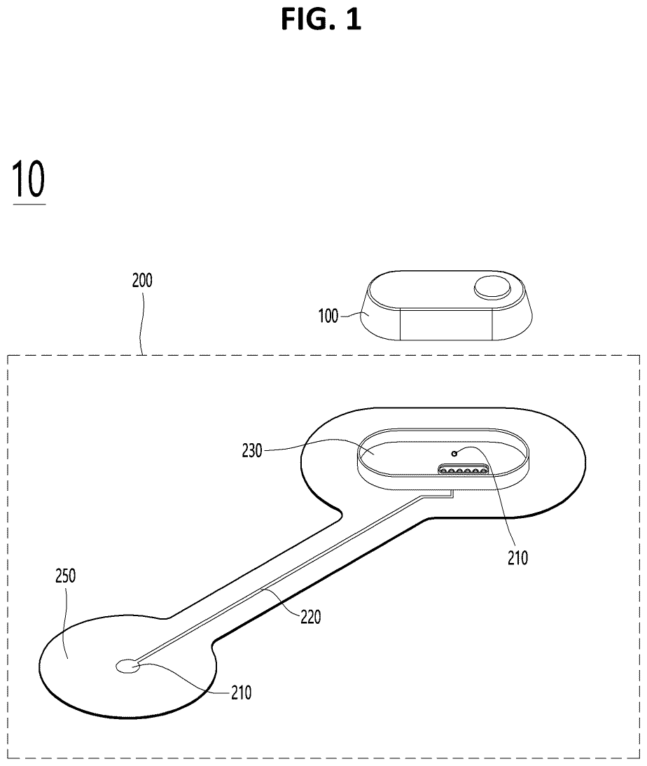

FIG. 1 shows a main body unit and an electrode unit according to one embodiment of the invention.

Referring to FIG. 1, a device 10 according to one embodiment of the invention may comprise a main body unit 100 and an electrode unit 200. Here, the electrode unit 200 according to one embodiment of the invention may include two or more electrodes 210, a circuit unit 220, and a pad 250. In the following, the detailed components of the electrode unit 200 will be discussed, and then the main body unit 100 will be discussed.

First, the electrode unit 200 according to one embodiment of the invention may include two or more electrodes 210 for acquiring a biosignal. Here, according to one embodiment of the invention, at least two electrodes 210 may be formed at a predetermined interval, and three electrodes including a reference electrode may preferably be formed. According to one embodiment of the invention, the electrodes 210 may form contact points with a patient's body to acquire a biosignal such as an electrocardiogram (ECG) signal, and the biosignal acquired from the electrodes 210 may be transmitted to the main body unit 100 through the circuit unit 220. For example, one of the two or more electrodes according to one embodiment of the invention may be formed at the bottom of a coupling unit 230 to acquire a biosignal.

Further, the electrode unit 200 according to one embodiment of the invention may include a circuit unit 220 for electrically connecting the electrodes 210 and the main body unit 100. Here, referring to FIG. 1, the circuit unit 220 according to one embodiment of the invention may include a circuit trace for transmitting the biosignal acquired from the electrodes 210 to the main body unit 100.

Furthermore, the electrode unit 200 according to one embodiment of the invention may include a pad 250. The pad 250 according to one embodiment of the invention may be arranged around the electrodes 210, and the electrodes 210 may form contact points with the patient's body when the pad 250 is attached to the patient, so that the electrodes 210 may acquire a biosignal. Here, the pad 250 according to one embodiment of the invention may include an attachment pad that is electrically connected to the circuit unit 220 and attached at a position appropriate for acquiring the patient's biosignal, and double-sided tape for attaching to the patient. For example, the biosignal according to one embodiment of the invention may be acquired by the electrodes 210 from the patient's body that is in contact with the attachment pad. Therefore, the pad 250 according to one embodiment of the invention may function to attach to the patient so that a biosignal may be acquired from the electrodes 210. Meanwhile, the attachment pad according to one embodiment of the invention may function to assist the electrodes 210 in acquiring the patient's biosignal by forming a contact point with the patient's body instead of the electrodes 210.

In addition, the coupling unit 230 according to one embodiment of the invention may function to allow the main body unit 100 and the electrode unit 200 to be detachably coupled. When the main body unit 100 and the electrode unit 200 are coupled by the coupling unit 230 according to one embodiment of the invention, the biosignal acquired from the electrode unit 200 may be transmitted to the main body unit 100, so that the main body unit 100 may store the biosignal.

Moreover, the biosignal according to one embodiment of the invention may include an electrocardiogram (ECG) signal. However, the biosignal according to the invention is not limited to an ECG signal, and may include various biosignals (e.g., blood oxygen saturation, heart rate, or body temperature) as long as the objects of the invention may be achieved.

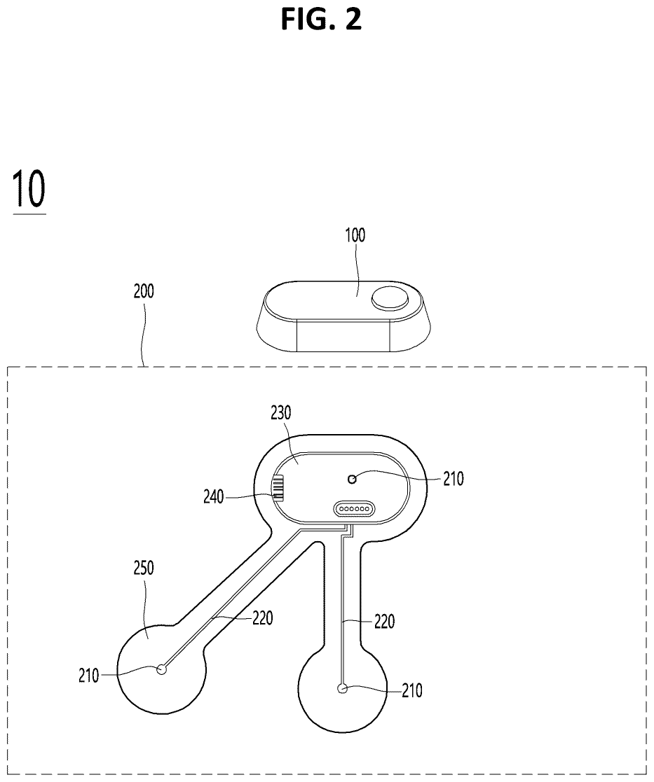

FIG. 2 shows the main body unit 100 and the electrode unit 200 according to one embodiment of the invention.

Referring to FIG. 2, for example, the electrode unit 200 according to one embodiment of the invention may include three electrodes. Here, referring to FIG. 2, one electrode may be arranged at the bottom of the coupling unit 230, and the other two electrodes may be arranged at appropriate positions for acquiring an ECG signal. Here, a channel according to one embodiment of the invention may be defined by one electrode (i.e., one unipolar electrode) using a Wilson central terminal (WCT) or two electrodes (i.e., a pair of electrodes), and when the electrode unit 200 includes three electrodes (e.g., electrodes arranged at positions associated with RA (Right Atrium), LA (Left Atrium), and LL (Left Leg) of 12 ECG leads in the chest area), biosignals related to two channels (i.e., a channel associated with the direction from RA to LA and a channel associated with the direction from RA to LL) may be acquired. Further, the channel according to one embodiment of the invention may be defined as a lead specified by two electrodes.

Meanwhile, it should be understood that one of the electrodes 210 according to the invention does not necessarily have to be arranged at the bottom of the coupling unit 230, but may be arranged at various points for achieving the objects of the invention.

An extension connection unit 240 may be further formed in the electrode unit 200 according to one embodiment of the invention. Specifically, an extension connection unit 240 electrically connected to the electrode unit 200 may be further formed in the electrode unit 200, and additional electrodes for acquiring the biosignal may be connected to the extension connection unit 240.

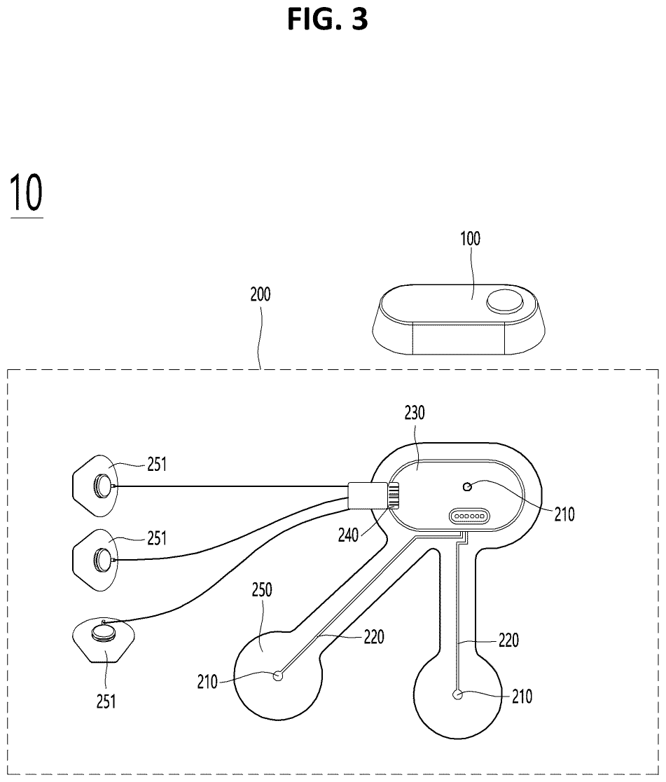

FIG. 3 shows the main body unit 100 and the electrode unit 200 according to one embodiment of the invention.

Referring to FIG. 3, additional electrodes for acquiring a biosignal may be connected to the extension connection unit 240 according to one embodiment of the invention, in which case the additional electrodes may be electrically connected to the extension connection unit through the circuit unit 220. Here, in order to acquire a biosignal from the additional electrodes, additional pads 251 may be arranged around the additional electrodes according to one embodiment of the invention, and the additional pads 251 may form contact points with the patient's body to acquire the biosignal. For example, when the biosignal according to one embodiment of the invention is an ECG signal, the additional electrodes may be arranged at least one position among V1, V2, V3, V4, V5, and V6 of the patient among 12 ECG leads, which are known to be appropriate for ECG signal acquisition, to acquire the ECG signal. In this case, it is possible to acquire the ECG signal in more diverse ways, so that the electrocardiogram signal is multilaterally identified and the patient's heart disease is accurately identified.

Next, the main body unit 100 according to one embodiment of the invention may include various components for operating the device 10, such as a battery, a printed circuit board assembly (PCBA), and a memory for storing the biosignal.

The main body unit 100 according to one embodiment of the invention may function to acquire unique information of the electrode unit 200 in response to being coupled to the electrode unit 200. Here, the unique information according to one embodiment of the invention may include information on the number or arrangement of the electrodes. For example, when the main body unit 100 and the electrode unit 200 according to one embodiment of the invention are coupled and the electrode unit 200 includes three electrodes 210, the unique information may include at least one of information indicating that the electrode unit 200 includes three electrodes and information indicating that the electrodes are arranged at positions associated with RA, LA, and LL, respectively.

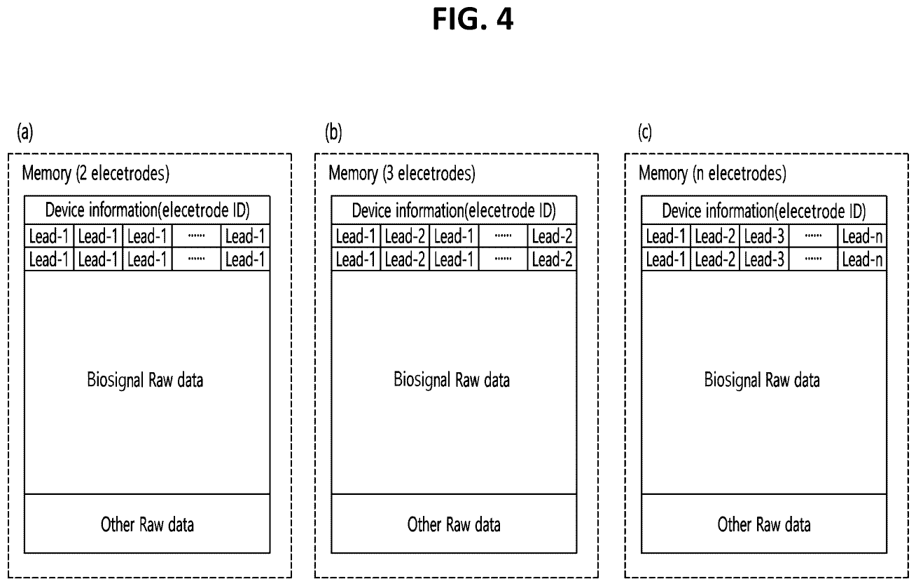

FIG. 4 illustratively shows a situation in which a biosignal is stored separately for each channel according to one embodiment of the invention.

Referring to FIG. 4, the main body unit 100 according to one embodiment of the invention may function to specify at least one channel implemented by the electrode unit 200 coupled to the main body unit 100, and store a biosignal acquired through the electrode unit 200 separately for each channel.

For example, referring to (a) of FIG. 4, when the electrode unit 200 according to one embodiment of the invention includes two electrodes respectively arranged at positions associated with RA and LL, there may be one channel implemented by the electrode unit 200, so that a single channel ECG signal may be acquired. Here, according to one embodiment of the invention, when the main body unit 100 and the electrode unit 200 are coupled, the main body unit 100 may acquire unique information indicating that the electrode unit 200 has two electrodes and the electrodes are arranged at positions associated with RA and LL, and the main body unit may store the single channel ECG signal.

Further, for example, referring to (b) of FIG. 4, when the electrode unit 200 according to one embodiment of the invention includes three electrodes respectively arranged at positions associated with RA, LA, and LL, and the main body unit 100 and the electrode unit 200 are coupled, the main body unit 100 may acquire unique information indicating that the electrode unit 200 has three electrodes and the electrodes are arranged at positions associated with RA, LA, and LL, and the main body unit may acquire biosignals from two channels (e.g., a channel associated with the direction from RA to LA and a channel associated with the direction from RA to LL).

When a single channel biosignal is stored, there is little need to separately store the biosignal since the biosignal is acquired at the same point. However, when biosignals are acquired from two channels as shown in (b) of FIG. 4, it may be difficult or impossible to monitor the acquired biosignals if the biosignals are not separated and organized. Therefore, as shown in (b) of FIG. 4, according to one embodiment of the invention, when biosignals are acquired from two channels, the main body unit 100 may function to store the acquired biosignals separately for each of the two channels with reference to the acquired unique information (e.g., information on the number or arrangement of the electrodes). For example, the main body unit 100 according to one embodiment of the invention may store the acquired biosignals alternately for each channel in chronological order. As a specific example, when biosignals a1, a2, and a3 are acquired sequentially from the electrodes related to the channel associated with the direction from RA to LA, and biosignals b1, b2, and b3 are acquired sequentially from the electrodes related to the channel associated with the direction from RA to LL, the main body unit 100 according to one embodiment of the invention may store the biosignals in the order of a1, b1, a2, b2, a3, and b3. According to one embodiment of the invention, when the biosignals are stored alternately for each channel as above, the biosignals may be stored for each channel in the main body unit 100 without undergoing separate additional processing, and the biosignals acquired from the electrodes related to various channels may be stored in a single data file, so that the structure of the device 10 may be simplified.

As another example, referring to (c) of FIG. 4, when the electrode unit 200 according to one embodiment of the invention includes three or more electrodes, the main body unit 100 may acquire information on the position and arrangement of the electrodes included in the electrode unit 200 as unique information as described above, and when biosignals are acquired, the main body unit 100 may store the biosignals separately for each channel as described above.

Meanwhile, the biosignal according to one embodiment of the invention is stored in an encrypted form, so that personal health information may be stored safely.

Finally, when a USB terminal is coupled to the main body unit 100 according to one embodiment of the invention and the main body unit is electrically connected to an external device (e.g., PC), the biosignal stored in the main body unit 100 may be transmitted to the PC. In this case, the transmitted biosignal according to one embodiment of the invention may be encrypted to safely protect personal health information.

Meanwhile, it should be understood that the term “patient” as used herein encompasses not only a patient with heart disease but also a patient who wants to check whether he/she has heart disease.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A device for monitoring a biosignal, the device comprising:
a main body unit; and
an electrode unit detachably coupled to the main body unit,
wherein the electrode unit includes two or more electrodes for acquiring the biosignal, and a circuit unit for electrically connecting the electrodes and the main body unit.

2. The device of claim 1, wherein the biosignal includes an electrocardiogram signal.

3. The device of claim 1, wherein a pad is arranged around the electrodes and attached to a patient, and the biosignal is acquired from the electrodes.

4. The device of claim 1, wherein the main body unit is configured to acquire unique information of the electrode unit in response to being coupled to the electrode unit.

5. The device of claim 4, wherein the unique information includes information on a number or arrangement of the electrodes.

6. The device of claim 1, wherein the main body unit is configured to specify at least one channel implemented by the electrode unit coupled to the main body unit, and store the biosignal acquired through the electrode unit separately for each channel.

7. The device of claim 1, wherein an extension connection unit electrically connected to the electrode unit is further formed in the electrode unit, and an additional electrode for acquiring the biosignal is capable of being connected to the extension connection unit.
